(19) United States
(12) Patent Application Publication
(10) Pub. No.: US 2022/0344059 A1
(43) Pub. Date: Oct. 27, 2022

(54) System, Method, and Computer Program Product for Detecting and Responding to Patient Neuromorbidity

(71) Applicant: UNIVERSITY OF PITTSBURGH - OF THE COMMONWEALTH SYSTEM OF HIGHER EDUCATION, Pittsburgh, PA (US)

(72) Inventors: Robert Clark, Pittsburgh, PA (US); Christopher Michael Horvat, Wexford, PA (US); Alicia Ka Win Au, Pittsburgh, PA (US); Amie Barda, Canfield, OH (US)

(21) Appl. No.: 17/760,558

(22) PCT Filed: Nov. 24, 2020
(86) PCT No.: PCT/US2020/061985

§ 371 Date: Mar. 15, 2022

Related U.S. Application Data
(60) Provisional application No. 62/940,977, filed on Nov. 27, 2019.

(51) Int. Cl. G16H 50/30; G16H 10/60; G06N 20/00

(52) U.S. Cl. CPC G16H 50/30; G16H 10/60; G06N 20/00

(57) ABSTRACT

Provided herein are systems, methods, and computer program products for use in detecting and responding to patient neuromorbidity. The method includes receiving patient cohort data from an electronic health record system and identifying features of the patient cohort data using a feature selection evaluating parameter. The method also includes training, using the features, a patient classification model configured to classify patients according to neuromorbidity risk. The method further includes receiving a patient dataset associated with a patient and generating, by inputting the patient dataset into the patient classification model, a patient classification of the patient comprising a probability of the patient developing a neuromorbidity over a time period. The method further includes, in response to the probability of the patient developing a neuromorbidity satisfying a predetermined threshold, transmitting an alert to a computing device associated with a physician, a nurse, and/or an advanced practice provider of deteriorating brain health.

### CROSS-REFERENCE TO RELATED APPLICATION

This application claims priority to U.S. Provisional Patent Application No. 62/940,977, filed Nov. 27, 2019, the disclosure of which is incorporated by reference herein in its entirety.

### BACKGROUND

### 1. Field

This disclosure relates generally to neuroscience and, in non-limiting embodiments, bio-digital methods and systems for monitoring brain health, and uses thereof, including detecting and responding to patient neuromorbidity.

### 2. Technical Considerations

The silent progression of new neurologic conditions and deterioration of brain health among critically ill patients admitted to hospitals is an emerging epidemic. This includes not only patients admitted to intensive care units (ICUs) with primary neurologic diagnoses, but also those where the development of neurologic morbidity (e.g., complication, problem caused by treatment), or neuromorbidity, is occult and unexpected. Critical illness-associated neuromorbidity spans the age spectrum from neonates to the elderly, occurs across gender and race, and is more prevalent in patients with pre-existing conditions. The estimated incidence of neuromorbidity ranges from 5-47% in critically ill children and adults, thus, impacting hundreds of thousands of patients in the U.S. annually. Often the progression of critical illness-associated neuromorbidity is “silent” and detected only after clinical manifestations become irreversible. Neuromorbidities range in severity from life-altering to life-threatening and include cognitive decline, delirium, chronic pain, seizures, intracranial hemorrhage, stroke, and minimally conscious (e.g., vegetative) state. Neuromorbidity can strike acutely, e.g., seizures, ischemic stroke, intracerebral hemorrhage, cerebral edema, and/or delirium, or in a more protracted fashion, e.g., neuromuscular weakness, and/or cognitive decline, and is typically permanent, endured throughout the remainder of a person's lifetime. No standard clinical tools exist to identify a patient's risk for neuromorbidity or for pragmatic real-time monitoring of a patient's brain health, in stark contrast to the heart, kidney, liver, and many other organs. There exists no standard of care methods for continuous, non-invasive monitoring of brain health or determining risk of neuromorbidity, in humans.

In further detail, mortality rates in pediatric intensive care units (PICUs), once as high as 11% in the 1980's, have declined over the past few decades, now reaching an unadjusted mortality rate of 1.3-5.0%. As survival in the modern pediatric intensive care unit (PICU) continues to increase, heightened emphasis has been placed on the impact of morbidity following critical illness. The contribution of neurological injury towards intensive care mortality is substantial. Neurological injury has a significant impact on mortality following admission to the PICU. Children in the intensive care unit (ICU) with neurologic diagnoses have more than 3 times the mortality of other patients (4.8% vs 1.5%). Neurological complications (e.g., seizure, stroke, intracerebral hemorrhage, or encephalopathy) develop in 12-33% of adult ICU patients admitted without an acute neurological disorder. While the incidence in pediatrics has not been well characterized, the presence of neurological diagnoses during pediatric critical illness is associated with use of mechanical ventilation, increased length of stay and morbidity.

There is a need in the art for an accurate, predictive system for identifying patients who are likely to develop a neuromorbidity and timely respond accordingly.

### SUMMARY

According to a non-limiting embodiment or aspect, provided is a computer-implemented method for detecting and responding to patient neuromorbidity. The method includes receiving, with at least one processor, patient cohort data from an electronic health record system. The method also includes identifying, with at least one processor, features of the patient cohort data using a feature selection evaluating parameter. The feature selection evaluating parameter includes at least one of the following: a first value, a last value, a minimum value, a maximum value, a difference between the minimum value and the maximum value, a difference between the first value and the last value, a change in value over a period of time, a slope of change over a period of time, or a combination thereof. The method further includes training, with at least one processor using the features, a patient classification model configured to classify patients according to neuromorbidity risk. The method further includes receiving, with at least one processor, a patient dataset associated with a patient and generating, with at least one processor by inputting the patient dataset into the patient classification model, a patient classification of the patient including a probability of the patient developing a neuromorbidity over a time period. The method further includes, in response to the probability of the patient developing a neuromorbidity satisfying a predetermined threshold, transmitting, with at least one processor, an alert to a computing device associated with at least one of the following: a physician, a nurse, an advanced practice provider of deteriorating brain health, or any combination thereof.

According to a non-limiting embodiment or aspect, provided is a system for detecting and responding to patient neuromorbidity. The system includes at least one server computer including at least one processor. The at least one server computer is programmed and/or configured to receive patient cohort data from an electronic health record system. The at least one server computer is also programmed and/or configured to identify features of the patient cohort data using a feature selection evaluating parameter including at least one of the following: a first value, a last value, a minimum value, a maximum value, a difference between the minimum value and the maximum value, a difference between the first value and the last value, a change in value over a period of time, a slope of change over a period of time, or a combination thereof. The at least one server computer is further programmed and/or configured to train, using the features, a patient classification model configured to classify patients according to neuromorbidity risk. The at least one server computer is further programmed and/or configured to receive a patient dataset associated with a patient. The at least one server computer is further programmed and/or configured to generate, by inputting the patient dataset into the patient classification model, a patient classification of the patient including a probability of the patient developing a neuromorbidity over a time period. The at least one server computer is further programmed and/or configured to, in response to the probability of the patient developing a neuromorbidity satisfying a predetermined threshold, transmit an alert to a computing device associated with at least one of the following: a physician, a nurse, an advanced practice provider of deteriorating brain health, or any combination thereof.

According to a non-limiting embodiment or aspect, provided is a computer program product for detecting and responding to patient neuromorbidity. The computer program product includes at least one non-transitory computer-readable medium including program instructions that, when executed by at least one processor, cause the at least one processor to receive patient cohort data from an electronic health record system. The program instructions further cause the at least one processor to identify features of the patient cohort data using a feature selection evaluating parameter including at least one of the following: a first value, a last value, a minimum value, a maximum value, a difference between the minimum value and the maximum value, a difference between the first value and the last value, a change in value over a period of time, a slope of change over a period of time, or a combination thereof. The program instructions further cause the at least one processor to train, using the features, a patient classification model configured to classify patients according to neuromorbidity risk. The program instructions further cause the at least one processor to receive a patient dataset associated with a patient. The program instructions further cause the at least one processor to generate, by inputting the patient dataset into the patient classification model, a patient classification of the patient including a probability of the patient developing a neuromorbidity over a time period. The program instructions further cause the at least one processor to, in response to the probability of the patient developing a neuromorbidity satisfying a predetermined threshold, transmit an alert to a computing device associated with at least one of the following: a physician, a nurse, an advanced practice provider of deteriorating brain health, or any combination thereof.

According to a non-limiting embodiment or aspect, provided is a method of medical treatment including receiving a patient classification of a patient from a computing device including a computer program product programmed and/or configured to operate a patient classification model, and treating the patient based on the patient classification.

According to a non-limiting embodiment or aspect, provided is a computer-implemented method for detecting and responding to patient neuromorbidity. The method includes receiving, with at least one processor, a patient dataset associated with a patient. The method also includes generating, with at least one processor by inputting the patient dataset into a patient classification model, a patient classification of the patient including a probability of the patient developing a neuromorbidity over a time period. The method further includes, in response to the probability of the patient developing a neuromorbidity satisfying a predetermined threshold, transmitting, with at least one processor, an alert to a computing device associated with at least one of the following: a physician, a nurse, an advanced practice provider of deteriorating brain health, or any combination thereof.

Other non-limiting embodiments or aspects are set forth in the following numbered clauses:

Clause 1: A computer-implemented method comprising: receiving, with at least one processor, patient cohort data from an electronic health record system; identifying, with at least one processor, features of the patient cohort data using a feature selection evaluating parameter comprising at least one of the following: a first value, a last value, a minimum value, a maximum value, a difference between the minimum value and the maximum value, a difference between the first value and the last value, a change in value over a period of time, a slope of change over a period of time, or a combination thereof; training, with at least one processor using the features, a patient classification model configured to classify patients according to neuromorbidity risk; receiving, with at least one processor, a patient dataset associated with a patient; generating, with at least one processor by inputting the patient dataset into the patient classification model, a patient classification of the patient comprising a probability of the patient developing a neuromorbidity over a time period; and, in response to the probability of the patient developing a neuromorbidity satisfying a predetermined threshold, transmitting, with at least one processor, an alert to a computing device associated with at least one of the following: a physician, a nurse, an advanced practice provider of deteriorating brain health, or any combination thereof.

Clause 2: The computer-implemented method of clause 1, wherein the patient dataset comprises at least one of the following: vital signs of the patient; drugs administered to the patient; pupillary response or reactivity of the patient; at least one bodily fluid parameter of the patient; a Glasgow coma scale score of the patient; a hemodynamic status and/or support; inflammation and/or invasive support; or any combination thereof.

Clause 3: The computer-implemented method of clause 1 or 2, wherein the patient classification model comprises a linear regression model or a logistic regression model.

Clause 4: The computer-implemented method of any of clauses 1-3, wherein the patient classification model comprises a machine-learning model executing at least one of the following techniques: Multivariate Adaptive Regression Splines (MARS); random forest; support vector machines; naïve Bayes; or any combination thereof.

Clause 5: The computer-implemented method of any of clauses 1-4, further comprising converting, with at least one processor, the features from time series data to vector space representations prior to training the patient classification model.

Clause 6: The computer-implemented method of any of clauses 1-5, further comprising, repeating, at a time interval, the following: receiving, with at least one processor, a new patient dataset from the record of the patient; generating, with at least one processor by inputting the new patient dataset into the patient classification model, a new patient classification of the patient comprising a new probability of the patient developing a neuromorbidity over a subsequent time period; and, in response to the new probability of the patient developing a neuromorbidity satisfying the predetermined threshold, transmitting, with at least one processor, the alert to the computing device associated with at least one of the following: the physician, the nurse, the advanced practice provider of deteriorating brain health, or any combination thereof.

Clause 7: The computer-implemented method of any of clauses 1-6, wherein the patient dataset comprises a value indicative of levels of at least one brain-specific biomarker, and wherein the patient classification is based at least partly on the value indicative of levels of the at least one brain-specific biomarker.

Clause 8: The computer-implemented method of any of clauses 1-7, wherein the at least one brain-specific biomarker comprises at least one of the following: ubiquitin carboxyl-terminal hydrolase L1 (UCH-L1), glial fibrillary acidic protein (GFAP), myelin basic protein (MBP), neuron specific enolase (NSE), S100b, neurofilament light chain (NFL), Tau, phosphorylated Tau (pTau), cleaved Tau (cTau), 150 kDa breakdown product of α-II-spectrin (SBDP150), or any combination thereof.

Clause 9: A system comprising at least one server computer including at least one processor, the at least one server computer programmed and/or configured to: receive patient cohort data from an electronic health record system; identify features of the patient cohort data using a feature selection evaluating parameter comprising at least one of the following: a first value, a last value, a minimum value, a maximum value, a difference between the minimum value and the maximum value, a difference between the first value and the last value, a change in value over a period of time, a slope of change over a period of time, or a combination thereof; train, using the features, a patient classification model configured to classify patients according to neuromorbidity risk; receive a patient dataset associated with a patient; generate, by inputting the patient dataset into the patient classification model, a patient classification of the patient comprising a probability of the patient developing a neuromorbidity over a time period; and, in response to the probability of the patient developing a neuromorbidity satisfying a predetermined threshold, transmit an alert to a computing device associated with at least one of the following: a physician, a nurse, an advanced practice provider of deteriorating brain health, or any combination thereof.

Clause 10: The system of clause 9, wherein the patient dataset comprises at least one of the following: vital signs of the patient; drugs administered to the patient; pupillary response or reactivity of the patient; at least one bodily fluid parameter of the patient; a Glasgow coma scale score of the patient; a hemodynamic status and/or support; inflammation and/or invasive support; or any combination thereof.

Clause 11: The system of clause 9 or 10, wherein the at least one server computer is further programmed and/or configured to convert the features from time series data to vector space representations prior to training the patient classification model.

Clause 12: The system of any of clauses 9-11, wherein the at least one server computer is further programmed and/or configured to repeat, at a time interval, the following: receiving a new patient dataset from the record of the patient; generating, by inputting the new patient dataset into the patient classification model, a new patient classification of the patient comprising a new probability of the patient developing a neuromorbidity over a subsequent time period; and in response to the new probability of the patient developing a neuromorbidity satisfying the predetermined threshold, transmitting the alert to the computing device associated with at least one of the following: the physician, the nurse, the advanced practice provider of deteriorating brain health, or any combination thereof.

Clause 13: The system of any of clauses 9-12, wherein the patient dataset comprises a value indicative of levels of at least one brain-specific biomarker, and wherein the patient classification is based at least partly on the value indicative of levels of the at least one brain-specific biomarker.

Clause 14: The system of any of clauses 9-13, wherein the at least one brain-specific biomarker comprises at least one of the following: ubiquitin carboxyl-terminal hydrolase L1 (UCH-L1), glial fibrillary acidic protein (GFAP), myelin basic protein (MBP), neuron specific enolase (NSE), S100b, neurofilament light chain (NFL), Tau, phosphorylated Tau (pTau), cleaved Tau (cTau), 150 kDa breakdown product of α-II-spectrin (SBDP150), or any combination thereof.

Clause 15: A computer program product comprising at least one non-transitory computer-readable medium including program instructions that, when executed by at least one processor, cause the at least one processor to: receive patient cohort data from an electronic health record system; identify features of the patient cohort data using a feature selection evaluating parameter comprising at least one of the following: a first value, a last value, a minimum value, a maximum value, a difference between the minimum value and the maximum value, a difference between the first value and the last value, a change in value over a period of time, a slope of change over a period of time, or a combination thereof; train, using the features, a patient classification model configured to classify patients according to neuromorbidity risk; receive a patient dataset associated with a patient; generate, by inputting the patient dataset into the patient classification model, a patient classification of the patient comprising a probability of the patient developing a neuromorbidity over a time period; and, in response to the probability of the patient developing a neuromorbidity satisfying a predetermined threshold, transmit an alert to a computing device associated with at least one of the following: a physician, a nurse, an advanced practice provider of deteriorating brain health, or any combination thereof.

Clause 16: The computer program product of clause 15, wherein the patient dataset comprises at least one of the following: vital signs of the patient; drugs administered to the patient; pupillary response or reactivity of the patient; at least one bodily fluid parameter of the patient; a Glasgow coma scale score of the patient; a hemodynamic status and/or support; inflammation and/or invasive support; or any combination thereof.

Clause 17: The computer program product of clause 15 or 16, wherein the program instructions further cause the at least one processor to convert the features from time series data to vector space representations prior to training the patient classification model.

Clause 18: The computer program product of any of clauses 15-17, wherein the program instructions further cause the at least one processor to repeat, at a time interval, the following: receiving a new patient dataset from the record of the patient; generating, by inputting the new patient dataset into the patient classification model, a new patient classification of the patient comprising a new probability of the patient developing a neuromorbidity over a subsequent time period; and, in response to the new probability of the patient developing a neuromorbidity satisfying the predetermined threshold, transmitting the alert to the computing device associated with at least one of the following: the physician, the nurse, the advanced practice provider of deteriorating brain health, or any combination thereof.

Clause 19: The computer program product of any of clauses 15-18, wherein the patient dataset comprises a value indicative of levels of at least one brain-specific biomarker, and wherein the patient classification is based at least partly on the value indicative of levels of the at least one brain-specific biomarker.

Clause 20: The computer program product of any of clauses 15-19, wherein the at least one brain-specific biomarker comprises at least one of the following: ubiquitin carboxyl-terminal hydrolase L1 (UCH-L1), glial fibrillary acidic protein (GFAP), myelin basic protein (MBP), neuron specific enolase (NSE), S100b, neurofilament light chain (NFL), Tau, phosphorylated Tau (pTau), cleaved Tau (cTau), 150 kDa breakdown product of α-II-spectrin (SBDP150), or any combination thereof.

Clause 21: A method of treating a patient having increased risk of development of a neromorbidity, comprising: receiving, from a computing device comprising the computer program product of any of clauses 15-20, the patient classification of the patient or the alert; and, increasing monitoring of the patient for development of the neuromorbidity and/or treating the patient for the neuromorbidity when the patient is classified as having increased risk of developing a neuromorbidity or an alert is transmitted indicating the patient as having increased risk of developing a neuromorbidity.

Clause 22: A computer-implemented method comprising: receiving, with at least one processor, a patient dataset associated with a patient; generating, with at least one processor by inputting the patient dataset into a patient classification model, a patient classification of the patient comprising a probability of the patient developing a neuromorbidity over a time period; and, in response to the probability of the patient developing a neuromorbidity satisfying a predetermined threshold, transmitting, with at least one processor, an alert to a computing device associated with at least one of the following: a physician, a nurse, an advanced practice provider of deteriorating brain health, or any combination thereof.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Additional advantages and details are explained in greater detail below with reference to the non-limiting, exemplary embodiments that are illustrated in the accompanying figures, in which:

FIG. 1 provides a schematic of a computer or computing device for use in systems and methods for detecting and responding to patient neuromorbidity according to non-limiting embodiments or aspects;

FIG. 2 depicts a schematic diagram of systems and methods for detecting and responding to patient neuromorbidity according to non-limiting embodiments or aspects;

FIG. 3 depicts a schematic diagram of a method of data extraction for systems for detecting and responding to patient neuromorbidity according to non-limiting embodiments or aspects;

FIG. 4 depicts a series of graphs representing a method of feature selection and model development for systems for detecting and responding to patient neuromorbidity according to non-limiting embodiments or aspects;

FIG. 5 depicts multiple data imputations with chained equations for use in systems and methods for detecting and responding to patient neuromorbidity according to non-limiting embodiments or aspects;

FIG. 6 depicts a schematic diagram of steps for advanced feature engineering for use in systems and methods for detecting and responding to patient neuromorbidity according to non-limiting embodiments or aspects;

FIG. 7 depicts a schematic diagram of risk calculation for automated alerting for use in systems and methods for detecting and responding to patient neuromorbidity according to non-limiting embodiments or aspects;

FIG. 8 depicts a schematic diagram of system deployment using Extract Transfer Load (ETL) from a Cerner Millennium database to a dedicated SQL server within an institutional firewall and a user interface with an ASP.NET web app viewable as an M-page in Cerner, according to non-limiting embodiments or aspects;

FIG. 9 depicts an evaluation of the predictive validity of a logistic regression model for detecting neurological morbidity in a subsequent 24 hour period, including graphs for Area Under the Receiver Operating Characteristic (AUROC) curve, positive predictive value, calibration, prediction density, accuracy, precision, and recall, according to non-limiting embodiments or aspects;

FIG. 10 depicts an evaluation of the predictive validity of a stepwise logistic regression model for detecting neurological morbidity in a subsequent 24 hour period, including AUROC, positive predictive value, calibration, prediction density, accuracy, precision, and recall, according to non-limiting embodiments or aspects;

FIG. 11 depicts an evaluation of the predictive validity of a Random Forest model for detecting neurological morbidity in a subsequent 24 hour period, including AUROC, positive predictive value, calibration, prediction density, accuracy, precision, and recall, according to non-limiting embodiments or aspects, suggesting a high-performing model;

FIG. 12 depicts multivariate adaptive regression splines, including clinically plausible U-shaped (e.g., heart rate), linear (e.g., GCS), and square-wave (e.g., benzodiazepine) splines, graphically represented with a y-axis corresponding to a relative contribution to risk of developing neuromorbidity, according to non-limiting embodiments or aspects;

FIG. 13 depicts an evaluation of predictive validity of the Multivariate Adaptive Regression Splines (MARS) model for detecting neurological morbidity in a subsequent 24 hour period, including AUROC, positive predictive value, calibration, prediction density, accuracy, precision, and recall, according to non-limiting embodiments or aspects, suggesting a high-performing model;

FIG. 14 depicts individual patient trajectories for serum UCH-L1, NSE, GFAP, and MBP displayed as fold-increase above Youden cutoff (Table 3) in patients where biomarkers were measured within 10 days before and/or after time-verified clinical diagnosis of neuromorbidity (n=16). Of the 16 patients, only two did not have a brain injury biomarker above the cutoff (patients N and P); and

FIG. 15 depicts a process diagram for a method of detecting and responding to patient neuromorbidity according to non-limiting embodiments or aspects.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions also refer to word forms, cognates and grammatical variants of those words or phrases.

As used herein, the terms “comprising,” “comprise” or “comprised,” and variations thereof, in reference to elements of an item, composition, apparatus, method, process, system, claim etc. are intended to be open-ended, meaning that the item, composition, apparatus, method, process, system, claim etc. includes those elements and other elements can be included and still fall within the scope/definition of the described item, composition, apparatus, method, process, system, claim etc. As used herein, “a” or “an” means one or more. As used herein “another” may mean at least a second or more.

As used herein, the terms “patient” or “subject” refer to members of the animal kingdom, including, but not limited to human beings.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

As used herein, the term “computing device” or “computer” may refer to one or more electronic devices configured to process data. A computing device may, in some examples, include the necessary components to receive, process, and output data, such as a display, a processor, a memory, an input device, and a network interface. A computing device may be a server, a mobile device, a desktop computer, a subsystem or integrated part of a genomic sequencer or sequence analyzer, and/or the like. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices.

As used herein, “interface” refers, in the context of programming and software modules, to the languages, codes and messages that programs or modules use to communicate with each other and to the hardware, and includes computer code or other data stored on a computer-readable medium that may be executed by a processor to facilitate the interaction between software modules. In some aspects of the methods and systems described herein, software modules, such as the variant calling module, the tumor phylogeny or modules and the machine learning modules are designed as separate software components, modules, or engines, with each requiring specific data input formats, and providing specific data output formats, and, in non-limiting examples, an interface may be used to facilitate such communication between components.

As used herein, the term “graphical user interface” or “GUI” refers to a generated display with which a user may interact, either directly or indirectly (e.g., through a keyboard, mouse, touchscreen, and/or the like).

As used herein, the term “electronic health record system” refers to a system including at least one computing device and at least one database for storing records of data corresponding to one or more patients, the data being representative of one or more attributes of a respective patient. As used herein, the term “patient cohort data” refers to data representative of one or more attributes of a plurality of patients.

As used herein, the term “satisfying” with respect to a threshold may include meeting and/or exceeding a threshold, which may include meeting or having a value less than a minimum-type threshold, and meeting or having a value greater than a maximum-type threshold.

As used herein, the terms “medical treatment” or “treating,” with respect to a patient, refers to taking one or more actions to improve the current and/or future condition of the patient. Medical treatment may include, but is not limited to, one or more of the following actions: administering a medication or other aid (e.g., oxygen) to the patient, modifying a level of monitoring of the patient, conducting one or more tests of the patient, conducting one or more surgical or reparative operations on the patient, providing one or more therapies or therapeutics to the patient, employing one or more medical devices for use on, in, or by the patient, modifying the position of the patient, increasing or reducing patient stimulation, modifying a diet of the patient, modifying an environment of the patient, and/or the like. As used herein, the term “neuromorbidity” may refer to a neurologic morbidity, including physical complications or problems caused by medical treatment, trauma, medical condition, or disease, such as an infection. Neuromorbidity may include, but is not limited to, cognitive decline, delirium, chronic pain, seizures, intracranial hemorrhage, stroke, impairment or loss of consciousness, seizures, ischemic stroke, intracerebral hemorrhage, cerebral edema, delirium, neuromuscular weakness, and/or the like.

Described systems and methods may be leveraged for automatically enacting one or more of the following events (e.g., in real-time with patient data creation): (i) detecting a neurological morbidity or neurological morbidities; (ii) alerting a physician, nurse, or advanced practice provider of the presence of neurological morbidity or neurological morbidities; (iii) alerting a physician, nurse, or advanced practice provider of deterioration of brain health; (iv) expeditiously retrieving electronic health record data that informs a physician, nurse, or advanced practice provider of the presence of a neurological morbidity or neurological morbidities; (v) expeditiously retrieving electronic health record data that informs a physician, nurse, or advanced practice provider of the state of brain health; (vi) expeditiously retrieving electronic health record data that informs a physician, nurse, or advanced practice provider of the deterioration in brain health; (vii) expeditiously processing electronic health record data to calculate the risk of developing a neurological morbidity or neurological morbidities; (viii) expeditiously processing electronic health record data to calculate deteriorating brain health; (ix) continuously monitoring for the development of a neurological morbidity or neurological morbidities; (x) continuously monitoring the state of brain health; (xi) continuously monitoring for deterioration in brain health; and/or (xii) expeditiously alerting a physician, nurse, or advanced practice provider of the presence of a neurological morbidity or neurological morbidities.

Described systems and methods provide for automated output or an on-demand output, from a classification model, representing the neuromorbidity risk classification for the patient. Said systems and methods are configurable to produce an automated warning output or an on-demand warning output, indicating that a patient is at increased risk of neuromorbidity. Neuromorbidity risk may be a measure of likelihood of a patient developing a neuromorbidity. The speed and accuracy of described systems and models greatly improves the ability of medical personnel to proactively assess and potentially treat patients with respect to predicted neuromorbidities, which may improve patient outcomes.

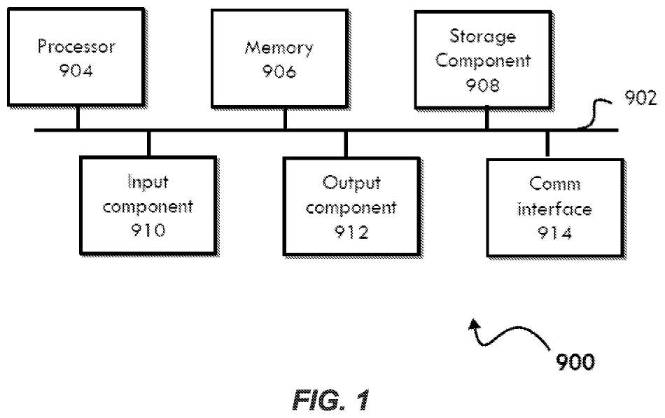

As shown in FIG. 1, provided is a computing device 900 for use in systems and methods for detecting and responding to patient neuromorbidity. Device 900 may include a bus 902, a processor 904, memory 906, a storage component 908, an input component 910, an output component 912, and a communication interface 914. Bus 902 may include a component that permits communication among the components of device 900. In some non-limiting embodiments, processor 904 may be implemented in hardware, firmware, or a combination of hardware and software. For example, processor 904 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 906 may include random access memory (RAM), read only memory (ROM), and/or another type of dynamic or static storage device (e.g., flash memory, magnetic memory, optical memory, etc.) that stores information and/or instructions for use by processor 904.

With continued reference to FIG. 1, storage component 908 may store information and/or software related to the operation and use of device 900. For example, storage component 908 may include a hard disk and/or another type of computer-readable medium (e.g., a magnetic disk, an optical disk, a magneto-optic disk, a solid state disk, cloud storage, etc.). Input component 910 may include a component that permits device 900 to receive information, such as via user input (e.g., a touch screen display, a keyboard, a keypad, a mouse, a button, a switch, a microphone, etc.). Output component 912 may include a component that provides output information from device 900 (e.g., a display, a speaker, one or more light-emitting diodes (LEDs), etc.). Communication interface 914 may include a transceiver-like component (e.g., a transceiver, a separate receiver and transmitter, etc.) that enables device 900 to communicate with other devices, such as via a wired connection, a wireless connection, or a combination of wired and wireless connections. Communication interface 914 may permit device 900 to receive information from another device and/or provide information to another device. For example, communication interface 914 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 900 may perform one or more processes described herein. Device 900 may perform these processes based on processor 904 executing software instructions stored by a computer-readable medium, such as memory 906 and/or storage component 908. A computer-readable medium may include any non-transitory memory device. A memory device includes memory space located inside of a single physical storage device or memory space spread across multiple physical storage devices. Software instructions may be read into memory 906 and/or storage component 908 from another computer-readable medium or from another device via communication interface 914. When executed, software instructions stored in memory 906 and/or storage component 908 may cause processor 904 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “programmed or configured,” as used herein, refers to an arrangement of software, hardware circuitry, or any combination thereof on one or more devices.

The computing device 900 can be configured to execute instructions for performing the computer-implemented tasks described herein. Software can be one or more of an operating system (e.g., a Windows™ based operating system), browser application, client application, server application, proxy application, on-line service provider application, and/or private network application. The software, modules, algorithms, interfaces, etc. can be implemented by utilizing any suitable computer language or analytical system (e.g., C\C++, UNIX SHELL SCRIPT, PERL, JAVA™, JAVASCRIPT, HTML/DHTML/XML, FLASH, WINDOWS, UNIX/LINUX, APACHE, RDBMS including ORACLE, INFORMIX, and MySQL, PYTHON, R, LISP, or PROLOG). Commercial software suites for implementation of machine learning, among the other functions and modules described herein, include free, open-source, and proprietary software, such as, without limitation, lifelines, SAS, MATLAB, among many others.

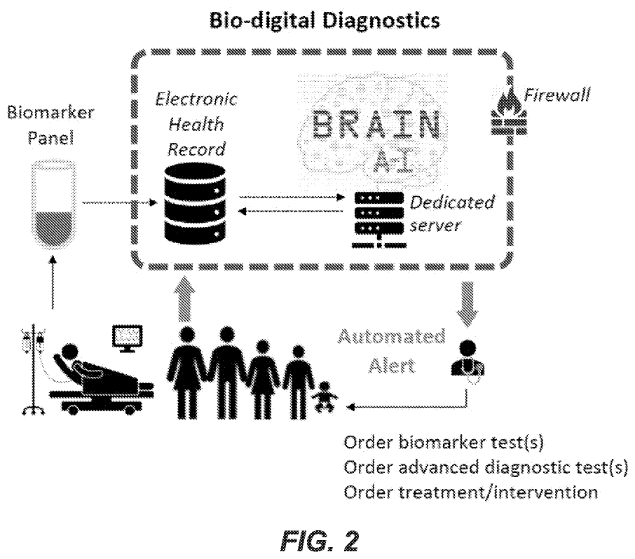

Referring to FIG. 2, provided herein is a bio-digital diagnostic process for identifying risk of neurologic morbidity, hereafter referred to as neuromorbidity, and monitoring brain health in humans. The process includes a novel digital electronic health record (EHR)-embedded, Bio-digital Rapid Alert to Identify Neuromorbidity (BRAIN) risk calculator encompassing 29 clinical variables in nine clinical domains beginning with letters A through I (BRAIN A-I) and a robust, biologically-verified decision support engine. BRAIN A-I includes a brain health monitoring risk stratification engine (BRAIN A-I bundle) to accurately identify subtle indicators of brain health deterioration in patients with non-obvious (e.g. traumatic brain injury (TBI)) brain injuries.

As determining risk and early identification are essential for prevention and/or treatment of neuromorbidity, BRAIN A-I is designed to fill this clinical gap. The BRAIN A-I bundle can be employed as a free-standing decision support tool for use in ICUs, hospitals, and/or health systems. The BRAIN A-I bundle can be employed in combination with brain-specific serum biomarkers in high-risk patients including critically ill patients with either non-neurologic or neurologic conditions to verify brain vulnerability and provide real-time clinical decision support to health care providers in ICUs, hospitals, and health systems.

In further detail to transform the way clinicians' monitor brain health for detection, prevention, and treatment of brain injury, described is a first-of-its-kind system also referred to herein as Bio-digital Rapid Alert to Identify Neuromorbidity (BRAIN). As described herein, electronic health record (EHR) data is combined with innovative bioinformatics and machine learning platforms to identify patients at risk for critical illness-associated neuromorbidity. Also described herein is an EHR-embedded point-of-care clinical risk calculator that incorporates up to 30 clinical variables in 9 clinical domains (A through I; Table 1). BRAIN A-I predicts new neuromorbidity within a time period (e.g., a 24-hour window) before the clinical event. For biological validation, provided are definitions of a panel of serum brain injury biomarkers including ubiquitin carboxyl-terminal hydrolase L1 (UCH-L1), glial fibrillary acidic protein (GFAP), and myelin basic protein (MBP) that are associated with new neuromorbidity for integration with BRAIN A-I. Importantly, provided is a live data pipeline bridging clinical and cloned research EHRs on a dedicated R-server, establishing the infrastructure necessary for implementing BRAIN as an integrated predictive analytic and decision-driving support tool (FIG. 2).

TABLE 1
BRAIN A-I risk calculator
Structured Data Elements

Age (days)
Behavior (level of consciousness; neuro-activity)
Cardiopulmonary resuscifation/coagulation
Drugs (opioid; barbiturate or benzodiazepine;
cisatracurium infusion)
Eyes (pupillary reactivity)
Fluids, electrolytes, nutrition (glucose; BUN;
z.creatinine; K⁺; min total CO₂; max total CO₂; hypo
Na⁺; hyper Na⁺)
Glasgow Coma Scale score
Hemodynamic status/support (z.SBP; z.heart rate;
epinephrine; norepinephrine; milrinone; dopamine;
dobutamine; vasopressin)
Inflammation/Invasive support (min T °; max T °;
mechanical ventilation; ECMO)
Abbreviations: BUN, blood urea nitrogen; ECMO, extracorporeal membrane oxygenation; SBP, systolic blood pressure; T °, temperature; z, z-scored

### Methods

A schematic overview of the bio-digital process is shown in FIG. 2, the below making reference thereto.

### Extraction of Input Data Elements

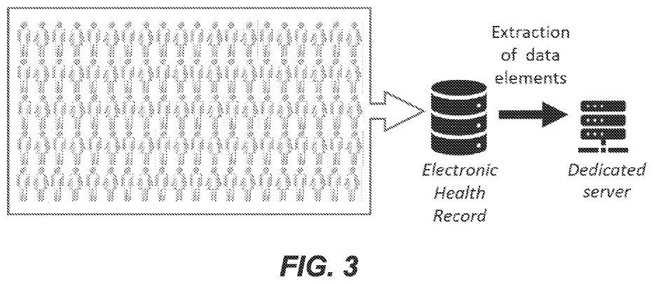

Model development proceeds with input data being obtained by interrogating an electronic health record (EHR) database (e.g., a cloned HER database) using a graphical user interface (GUI). The GUI may author structured query language (SQL) code. Data may be extracted and uploaded to a dedicated server. For example, the data may be extracted as XML files and uploaded to a secure server running R statistical analysis software version 3.4.1 (www.r-project.org) within an institutional firewall for cleaning and curation (see FIG. 3). A Cerner Millennium database may be used as a data pipeline, and Informatica Change Data Capture may be used to extract, transform, and load (ETL) data to the dedicated server. The dedicated server hosting R statistical analysis software is used to run patient classification models, including BRAIN A-I models, on the most recently available data and generate risk stratification in the form of probabilities. Analysis may be performed in real-time with the updating of the EHR. As input data are updated more frequently in patient care areas with higher patient acuity, high acuity hospital zones such as intensive care units (ICUs), scores will be refreshed more frequently as data are available. It will be appreciated that effectively real-time evaluation of high-refresh patient data yields more accurate and timely critical patient forecasts.

### Data Cleaning, Missingness Analysis, and Imputation

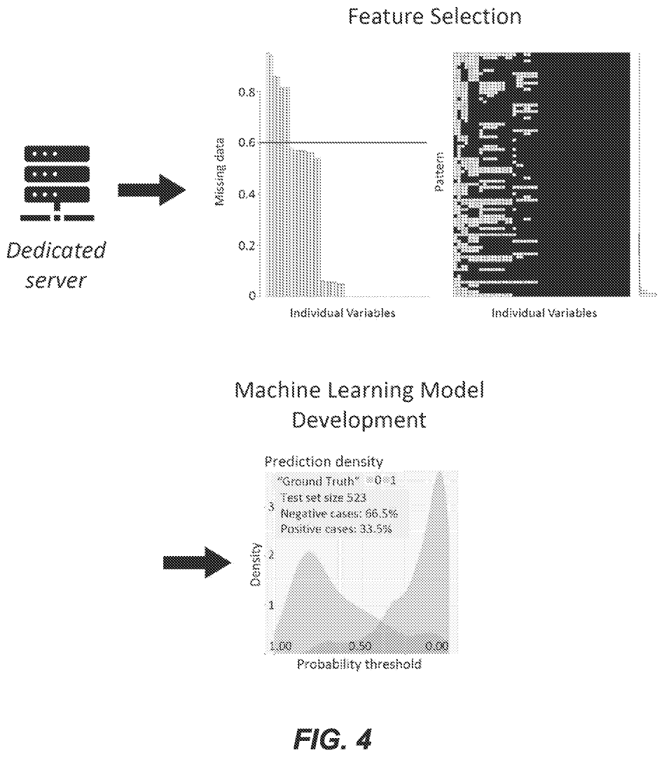

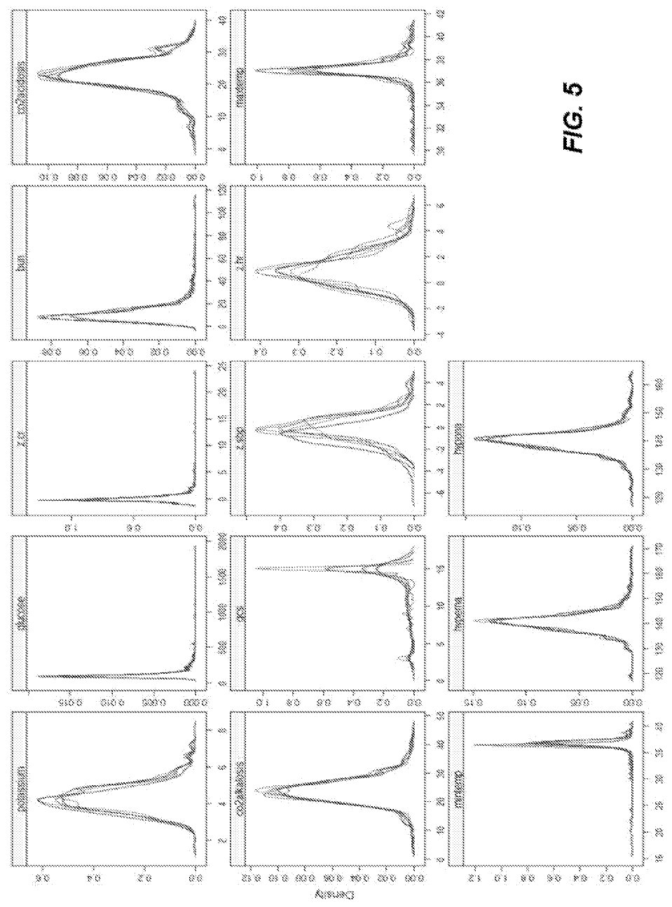

For pediatric applications, age-dependent, continuous variables (e.g., heart rate, systolic blood pressure, and creatinine) are transformed to z scores based on distributions of discharge vital signs of control patients, e.g., those who are not admitted to an ICU. Continuous variables are stratified according to four age categories: (1) less than 1 year; (2) 1 year to less than 4 years; (3) 4 years to 12 years; and (4) greater than 12 years. Variables with less than 60% of observations missing may be retained for initial modeling (see FIG. 4). Given that candidate variables span clinical observations that include vital signs, laboratory tests, medications, and nursing assessments, it may be assumed that existing observations are sufficient to provide an estimate of missing data. Additionally, as patient data may contain both continuous and categorical variables reflecting overlapping elements of patient state, the potential for both complex interaction terms, collinearity, and quasi-complete separation of the data, may be assumed when generalized linear methods (e.g., linear or logistic regression) are used for imputation. Multiple imputation with chained equations using classification and regression trees may be used to impute missing values over five datasets (e.g., over 5 iterations) (see FIG. 5). The distributions of imputed data may be compared to non-imputed-data with overlapping density plots to assess for deviation.

### Variable Selection

Bio-digital input variables are chosen on the basis of clinical expertise and include, but are not limited to: age, nursing assessment of neurologic activity; nursing assessment of level of consciousness; arterial, venous or capillary pCO₂ (PaCO₂, PvCO₂, PcCO₂); admission International Classification of Diseases version 10 code for cardiac arrest (CPR); prothrombin time (PT); activated partial thromboplastin time (PTT); administration of a cisatracurium infusion; administration of any neuromuscular blockade agent (cisatracurium, rocuronium, or vecuronium); administration of an intravenous benzodiazepine or barbiturate (lorazepam, midazolam, diazepam, pentobarbital; phenobarbital); administration of an intravenous opioid (morphine, fentanyl, hydromorphone, or methadone); administration of other sedative mediation (ketamine, etomidate, or propofol); pupillary response; serum creatinine (Cr); serum blood urea nitrogen (BUN); serum bicarbonate (HCOs); serum potassium (K); serum glucose; arterial, venous or capillary pH; serum sodium (Na); Glasgow Coma Scale score (GCS); administration of dopamine; administration of dobutamine; administration of epinephrine; administration of norepinephrine; administration of milrinone; administration of vasopressin; presence of extracorporeal life support (ECMO); serum lactate; mean, diastolic, systolic blood pressure (MBP, DBP, SBP); heart rate (HR); oxygen saturation (Oxy); temperature (Temp); presence of mechanical ventilation (MV); and presence of either an arterial or central venous catheter.

Variable selection may be customized based on individual hospital, healthcare system, or institution. Variable selection may be reevaluated periodically to determine if practices or patient characteristics change with time.

### Model Construction

Models may be custom-generated and determined based on individual hospital, healthcare system, or institutional data. Models may be reevaluated periodically to determine if practices or patient characteristics change over time.

Model conceptualization may adhere to the Littenberg framework for development of clinical decision support tools, which considers the clinical and technical plausibility of the tool, as well as process outcomes, patient outcomes, and eventual societal outcomes addressed by the tool.

An individual hospital, healthcare system, or institution cohort may be divided randomly 3:1 into development and validation datasets, to ensure comparable distributions of the outcome between train and test data. Summary data are presented with descriptive statistics. Unique models are fit in the development data and performances are assessed in the validation data. Models include, but are not be limited to, multivariable generalized linear models with a logit link function (logistic regression model), parsimonious multivariable generalized linear models with logit link function with variable selection performed using forward-backward stepwise selection based on Akaike Information Criterion (stepwise logistic regression model). Models are generated using methods that include, but are not limited to, logistic regression, stepwise logistic regression, Multivariate Adaptive Regression Splines (MARS) (e.g., with 10-fold cross validation), and random forest models (e.g., with 1000 trees). Model performances are assessed by calculating the area under the receiver operating characteristic (AUROC) and the area under the precision recall curve (AUPRC). Models are compared using the DeLong test to assess for significant differences in AUROCs and using Student's t test to compare average precision. Nonparametric continuous data are compared with the Wilcoxon rank sum test, and proportion data are compared with the Chi squared test. By way of example, analyses may be performed in R version 3.5.3 (www.r-project.org) using RStudio version 1.2.1335 (rstudio.com) and include the following packages: plyr (version 1.8.4), dplyr (0.8.1), earth (5.1.1), randomForest (4.6.14), classifierplots (1.3.3), precrec (0.10.1), and mice (3.5.0) (plyr: Tools for Splitting, Applying and Combining Data. 2016; dplyr: A Grammar of Data Manipulation. 2019; wrapper SMD from mda:mars by TH and RTUAMF utilities with TL leaps. earth: Multivariate Adaptive Regression Splines. 2019; original by LB and A, Wiener R port by AL and M. randomForest: Breiman and Cutler's Random Forests for Classification and Regression. 2018; classifierplots: Generates a Visualization of Classifier Performance as a Grid of Diagnostic Plots. 2017; precrec: Calculate Accurate Precision-Recall and ROC (Receiver Operator Characteristics) Curves. 2019; and mice: Multivariate Imputation by Chained Equations. 2019, all at rdrr.io/.

### Advanced Feature Engineering

For predictive models to more completely leverage the content of the EHR, the temporality of data may be incorporated into model features. Therefore, feature engineering and model development strategies that rely on conversion of time series data to vector space representations of patient state (Hauskrecht M, Batal I, Valko M, Visweswaran S, Cooper G F, Clermont G. Outlier detection for patient monitoring and alerting. J Biomed Inform 2013; 46:47-55) may be employed.

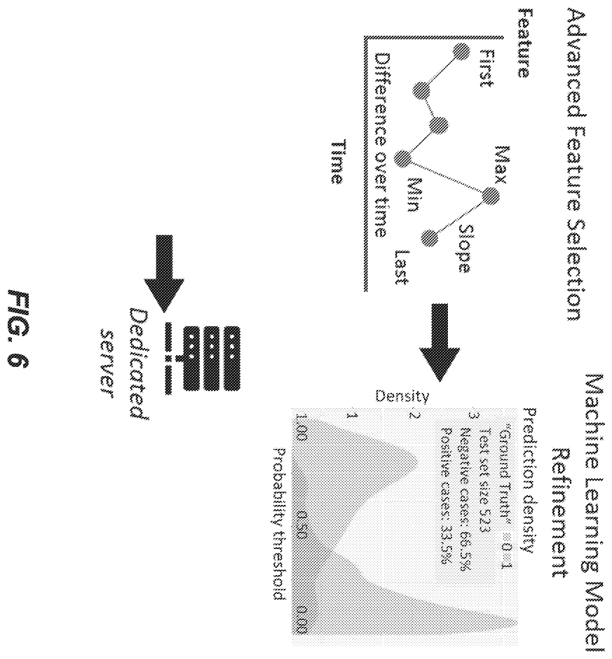

Vector space representation of patient state generated features are developed based on the type of origin data (see FIG. 6). Categorical laboratory values are considered according to first value, second to last value, last value, time since last value, and indicators of the test order status. Continuous variable features are generated by summarizing time-dependent aspects of the data, including the absolute difference between two measurements, the slope and percent change between two measurements, and comparisons between most recent values with previous, apex, nadir, and baseline values (Hauskrecht M, Batal I, Valko M, Visweswaran S, Cooper G F, Clermont G. Outlier detection for patient monitoring and alerting. J Biomed Inform 2013; 46:47-55).

In addition to feature-based vectorization, deep learning models for classification of time series data may be employed, including but not limited to long-short term memory networks and numerous related variants to the classification of time series data sets. Deep learning approaches are compared to vector space approaches in terms of both accuracy and training time.

### Advance Feature Selection

Advanced features for selection include but are not limited to: AdmitDiagnosisCodeCategory, PT__min, PTT_min, PT_last, PT_per_change_last_min, PT_diff_last_min, PT_max, PTT_per_change_last_min, PTT_diff_last_min, PTT_last, PTT_max, PedsComa_min, PT_sec_last, PedsComa_per_change_last_min, PTT_sec_last, PedsComa_diff_last_min, PT_diff_last_two, PT_per_change_last_two, WBC_diff_last_max, Platelets_diff_last_max, WBC_per_change_last_max, Platelets_per_change_last_max, CO₂_max, Glucose_min, Platelets_sec_last, Platelets_per_change_last_two, CO₂_slope_last_first, Platelets__diff_last_min, Platelets_per_change_last_min, WBC_min, WBC_diff_last_min, WBC_per_change_last_min, CO₂_first, Platelets_min, CO₂_per_change_last_first, CO₂_sec_last, Glucose_sec_last, PT_slope_last_first, PaO₂_max, PTT_first, PT_first, Glucose_last, pH_max, Platelets_max, LOS, BUN_diff_last_max, pH_diff_last_max, PTT_slope_last_first, CO₂_diff_last_first, pH_per_change_last_max, BUN_max, Glucose_diff_last_two, PT_slope_last_max, PaCO₂_min, WBC_max, K_slope_last_two, K_per_change_last_max, BUN_per_change_last_max, CO₂_per_change_last_max, Glucose_slope_last_two, CO₂_diff_last_max, K_diff_last_max, K_sec_last, K_per_change_last_two, Glucose_slope_last_first, CO₂_per_change_last_min, WBC__last, K__diff_last_two, PaO₂__diff_last_max, Glucose_per_change_last_two, PaCO₂_per_change_last_max, Glucose__per_change_last_max, Glucose_diff_last_max, Platelets_first, PaCO₂_per_change_last_min, PaO₂_per_change_last_min, PaO₂_per_change_last_max, K_slope_last_first, Glucose_diff_last_min, Platelets_slope_last_first, Platelets_per_change_last_first, CO₂_slope_last_two, PTT_slope_last_max, PaO₂_last, PaO₂_diff_last_min, PaCO₂_diff_last_max, PaCO₂_diff_last_min, K__max, Glucose__slope_last_max, Glucose_per_change_last_min, BUN_per_change_last_min, BUN_diff_last_min, Glucose_slope_last_min, Cr_slope_last_first, K_per_change_last_min, K_min, CO₂__last, BUN_slope_last_first, CO₂_diff_last_min, Glucose_first, K_diff_last_min, CO₂_min, PaCO₂_slope_last_first, K_slope_last_min, pH_per_change_last_min, pH_diff_last_min, Glucose_diff_last_first, HR_slope_last_first, BUN_min, BUN_slope_last_min, PaCO₂_first, PaO₂_slope_last_first, SpO_slope_last_first, BUN_first, pH_min, Glucose_per_change_last_first, BUN_diff_last_first, BUN_slope_last_max, PaCO₂_slope_last_max, K_slope_last_max, pH_slope_last_min, PedsComa_per_change_last_first, pH_sec_last, CO₂_slope_last_max, pH_per_change_last_two, pH_slope_last_max, Cr_diff_last_first, Cr_first, CO₂_slope_last_min, PupilReaction_both_nonreactive_count, PedsComa__diff_last_first, pH_slope_last_two, pH_slope_last_first, Cr_max, SBP_max, Cr_per_change_last_max, PvCO₂__max, Cr_per_change_last_min, Cr_diff_last_min, PedsComa_slope_last_first, Cr__diff_last_max, PedsComa_first, PvCO₂_diff_last_max, pH_first, Cr_slope_last_max, pH_diff_last_first, pH_per_change_last_first, PvCO₂_per_change_last_max, DBP_slope_last_first, MeanBP_slope_last_first, HR_slope_last_max, MeanBP_max, SpO_first, HR_min, Temp_slope_last_first, MeanBP_diff_last_max, Temp__per_change_last_min, SBP_slope_last_first, DBP_slope_last_min, PvCO₂_min, SBP__diff_last_max, Lactate_max, Cr__last, Lactate_diff_last_max, Cr_slope_last_min, MeanBP_slope_last_min, Temp_diff_last_min, DBP_max, PvCO₂_last, PvCO₂_slope_last_first, SpO_per_change_last_max, Lactate_per_change_last_max, HR_first, PupilReaction_first, Ventilator_flag, SpO_last, PvCO₂_first, Lactate_min, SpO_diff_last_max, Temp_slope_last_max, SpO_sec_last, MeanBP_per_change_last_max, Temp_max, PvCO₂_slope_last_max, AdmitUnit, Temp_diff_last_first, HR_slope_last_min, SpO_min, PvCO₂_per_change_last_first, SBP_per_change_last_max, PvCO₂_slope_last_min, Temp_per_change_last_first, DBP__diff_last_max, SpO_per_change_last_first, PvCO₂_diff_last_first, PvCO₂_sec_last, SBP_per_change_last_min, Temp_min, PedsComa_sec_last, SBP_slope_last_max, PedsComa__slope_last_min, DBP_slope_last_max, Lactate_first, SBP_slope_last_min, SpO_per_change_last_min, Temp_first, Lactate_diff_last_first, SBP_min, Lactate_slope_last_max, MeanBP_slope_last_max, Lactate__slope_last_first, PupilReaction_sec_last, PupilReaction_last, DBP_per_change_last_max, PedsComa_slope_last_two, SBP_diff_last_min, OxyPer_max, PedsComa_diff_last_two, PedsComa__per_change_last_two, SpO_slope_last_max, SpO_diff_last_first, SpO_per_change_last_two, PedsComa_per_change_last_max, DBP_per_change_last_min, MeanBP_min, HR_per_change_last_min, MeanBP_per_change_last_min, PedsComa_slope_last_max, SpO_slope_last_two, Temp_per_change_last_max, SpO_diff_last_min, Temp_diff_last_max, PedsComa_last, CPR_flag, HR_diff_last_first, OxyPer_first, DBP_min, SBP__first, PupilReaction_one_nonreactive_count, PedsComa_diff_last_max, HR_per_change_last_first, HR_slope_last_two, AgeDays, Temp_slope_last_min, Cancer_flag, MeanBP__diff_last_min, DBP__diff_last_min, CI_slope_last_first, MeanBP_slope_last_two, CI_slope_last_max, CI_per_change_last_max, CI__min, HR_sec_last, SpO_max, SpO_diff_last_two, HR_last, PedsComa_max, OxyPer_per_change_last_min, OxyPer_last, HR_diff_last_min, Temp_sec_last, OxyPer_diff_last_min, CI_diff_last_max, OxyPer_sec_last, PcCO₂_slope_last_max, CI_max, Temp_last, OxyPer_diff_last_max, CI_per_change_last_min, OxyPer_per_change_last_max, CI_diff_last_min, HR_max, HR_per_change_last_max, DBP_first, Race, OxyPer_min, MeanBP__first, MeanBP_per_change_last_two, MeanBP__diff_last_two, Sex, BUN_last, BUN_sec_last, BUN_diff_last_two, BUN_per_change_last_two, BUN_per_change_last_first, BUN_slope_last_two, CI__last, CI_sec_last, CI_first, CI_diff_last_two, CI_diff_last_first, CI_per_change_last_two, CI_per_change_last_first, CI_slope_last_two, CI_slope_last_min, CO₂_diff_last_two, CO₂_per_change_last_two, Cr_sec_last, Cr_min, Cr_diff_last_two, Cr_per_change_last_two, Cr_per_change_last_first, Cr_slope_last_two, DBP_last, DBP_sec_last, DBP_diff_last_two, DBP_diff_last_first, DBP_per_change_last_two, DBP_per_change_last_first, DBP_slope_last_two, Glucose__max, HR_diff_last_two, HR_diff_last_max, HR__per_change_last_two, K__last, K__first, K_diff_last_first, K_per_change_last_first, Lactate_last, Lactate_sec_last, Lactate__diff_last_two, Lactate_diff_last_min, Lactate_per_change_last_two, Lactate_per_change_last_min, Lactate_per_change_last_first, Lactate_slope_last_two, Lactate_slope_last_min, MeanBP__last, MeanBP_sec_last, MeanBP_diff_last_first, MeanBP_per_change_last_first, OxyPer__diff_last_two, OxyPer__diff_last_first, OxyPer_per_change_ast_two, OxyPer_per_change_last_first, OxyPer_slope_last_two, OxyPer_slope_last_min, OxyPer_slope_last_max, OxyPer_slope_last_first, PaCO₂_last, PaCO₂_sec_last, PaCO₂_max, PaCO₂_diff_last_two, PaCO₂_diff_last_first, PaCO₂_per_change_last_two, PaCO₂_per_change_last_first, PaCO₂_slope_last_two, PaCO₂_slope_last_min, PaO₂_sec_last, PaO₂_min, PaO₂__first, PaO₂_diff_last_two, PaO₂_diff_last_first, PaO₂_per_change_last_two, PaO₂_per_change_last_first, PaO₂_slope_last_two, PaO₂_slope_last_min, PaO₂_slope_last_max, PcCO₂_last, PcCO₂__sec_last, PcCO₂__min, PcCO₂_max, PcCO₂_first, PcCO₂_diff_last_two, PcCO₂_diff_last_min, PcCO₂_diff_last_max, PcCO₂_diff_last_first, PcCO₂_per_change_last_two, PcCO₂_per_change_last_min, PcCO₂_per_change_last_max, PcCO₂_per_change_last_first, PcCO₂_slope_last_two, PcCO₂_slope_last_min, PcCO₂_slope_last_first, pH_last, pH_diff_last_two, Platelets_last, Platelets_diff_last_two, Platelets_diff_last_first, Platelets_slope_last_two, Platelets_slope_last_min, Platelets_slope_last_max, PT_diff_last_max, PT_diff_last_first, PT_per_change_last_max, PT_per_change_last_first, PT_slope_last_two, PT__slope_last_min, PTT__diff_last_two, PTT__diff_last_max, PTT__diff_last_first, PTT__per_change_last_two, PTT_per_change_last_max, PTT_per_change_last_first, PTT__slope_last_two, PTT_slope_last_min, PvCO₂_diff_last_two, PvCO₂_diff_last_min, PvCO₂_per_change_last_two, PvCO₂_per_change_last_min, PvCO₂_slope_last_two, SBP_last, SBP_sec_last, SBP_diff_last_two, SBP_diff_last_first, SBP_per_change_last_two, SBP_per_change_last_first, SBP_slope_last_two, SpO_slope_last_min, Temp_difflast_two, Temp_per_change_last_two, Temp_slope_last_two, WBC_sec_last, WBC_first, WBC__diff_last_two, WBC_diff_last_first, WBC_per_change_last_two, WBC_per_change_last_first, WBC_slope_last_two, WBC_slope_last_min, WBC__slope_last_max, WBC_slope_last_first.

*Key: AdmitDiagnosisCodeCategory, admission diagnosis code category; PT, prothrombin time; min, minimum value; max, maximum value; PTT, partial thromboplastin time; diff_last_max, difference between last and maximum value; last, last value; sec_last, second to last value; PedsComa, Glasgow Coma Scale score; per_change_last_min, percent change from last to minimum value; diff_last_min, difference between last and minimum value; diff_last_two, difference between last two values; WBC, white blood cell count; per_change_last_max, percent change from last to maximum value; CO₂, carbon dioxide; per_change_last_two, percent change between last two values; slope_last_first, slope defined by last and first value; first, first value; per_change_last_first, percent change between last and first value; LOS, length of stay; BUN, blood urea nitrogen; diff_last_first, difference between last and first value; slope_last_max, slope defined by last and maximum value; PaCO₂, partial pressure of arterial carbon dioxide; K, potassium; slope_last_two, slope defined by last two values; PaO₂, partial pressure of arterial oxygen; slope_last_min, slope defined by last and minimum value; Cr, creatinine; SpO, peripheral oxygen saturation; PvCO₂, partial pressure of venous carbon dioxide; DBP, diastolic blood pressure; MeanBP, mean blood pressure; HR, heart rate; Temp, temperature; SBP, systolic blood pressure; PupilReaction, pupillary reaction; Ventilator_flag, received mechanical ventilation; AdmitUnit, admission unit; OxyPer, percent inspired oxygen; CPR_flag, received cardiopulmonary resuscitation; one_nonreactive_count, one nonreactive pupil; AgeDays, age in days; Cancer_flag, cancer diagnosis; CI, chloride; PcCO₂, partial pressure of capillary carbon dioxide.

A feature selection evaluating parameter may include one or more attributes (e.g., statistical attribute) of features that may be used for training a patient classification model (e.g., a deterministic or stochastic model receiving an input of patient data and generating an output of a qualification or quantification of likely patient outcome) and evaluate features associated with or determinative of a patient classification. A feature selection evaluating parameter may include, but is not limited to: a first value, a last value, a minimum value, a maximum value, a difference between the minimum value and the maximum value, a difference between the first value and the last value, a change in value over a period of time, a slope of change over a period of time, or a combination thereof.

### Risk Calculation Output

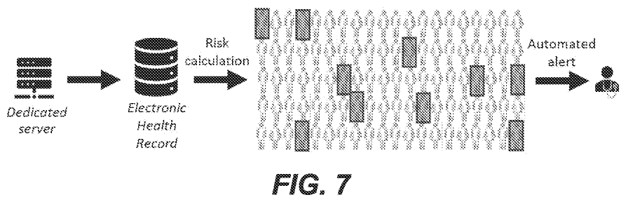

Risk of neurological deterioration is modeled with varying time horizons (e.g., time periods) and model selection is based on predictive performance and institutional circumstances, with attention to the five rights of clinical decision support. Model output is provided a probability of neurological deterioration following the selected time horizon (e.g., a 20% probability of deterioration in 6 hours). Automated alerts are sent to healthcare professionals, such as first-responder providers, including physicians, advanced practice providers, and/or nurses (see FIG. 7).

Integration with EHR for Generation of Automated Alert

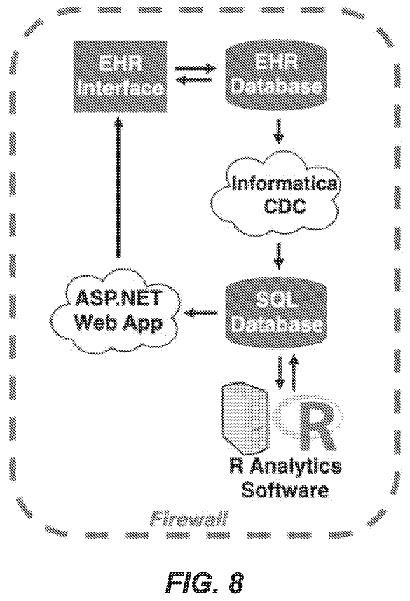

The previously described deployment data pipeline may be used for model deployment. The Informatica Change Data Capture tool may be used for real-time model output. Other machine-learning model deployment platforms, such as Epic's Cognitive Computing platform, can also be leveraged for model deployment (see FIG. 8).

### Results

BRAIN A-I models were constructed using a retrospective dataset that included all discharged patients with a PICU admission at our quaternary center between Jan. 1, 2017, and Dec. 31, 2017, (n=2,089), a subset of 23,179 PICU admissions discharged between Jan. 1, 2007, and Dec. 31, 2018, with acuity-adjustment for risk of mortality. Up to 30 clinical variables from 9 clinical domains were initially evaluated with candidate variables included only if <60% of observations were missing. The 30 clinical variables included minimum temperature, maximum temperature, Glasgow Coma Scale score, systolic blood pressure, age, heart rate, glucose, blood urea nitrogen, creatinine, level of consciousness, opioid, hypematremia, hyponatremia, potassium, minimum total CO₂, maximum total CO₂, benzodiazepine or barbiturate, neurological activity, mechanical ventilation, milrinone infusion, pupillary response, epinephrine infusion, norepinephrine infusion, dopamine infusion, dobutamine infusion, vasopressin infusion, cisatracurium infusion, cardiopulmonary resuscitation, extracorporeal membrane oxygenation, central venous or arterial catheter. Models were generated using logistic regression as well as machine learning techniques (MARS and Random Forest) for prediction of neuromorbidity within 24 h using an EHR-derived, computable composite outcome definition. In brief, the definition is based on salient clinical studies and medication administration strongly associated with significant neurologic disease. The definition encompasses two categories of neurologic morbidity: a structural or electrographic category identified based on documentation of neuroimaging that includes brain magnetic resonance imaging (MRI) or head computerized tomography (CT) scan or electroencephalography (EEG); and a behavioral category defined as a documented consult by the study institution's mental health team and administration of an anti-delirium (dexmedetomidine) or anti-psychotic (olanzapine or haloperidol) medication. We selected this outcome based on a favorable sensitivity and specificity of 68% and 99%, respectively. Controls were defined as encounters that did not meet the computable composite definition of neuromorbidity during hospitalization. Of the 2,089 children included in the prototype analysis, 700 (33.5%) met the computable composite definition of neuromorbidity.

Performance of prototype models were assessed by AUROCs and AUPRCs and are presented in Table 2, along with results of significance testing for model comparisons using DeLong's test. MARS and Random Forest showed excellent performance characteristics and were comparable. The GCS score, the most commonly used and “default” clinical assessment tool for classifying neurologic acuity, and Pediatric Risk of Mortality IV (PRISM IV), a contemporary multi-system score for mortality risk that includes neurological variables, both performed poorly relative to neurologic-focused BRAIN A-I models.

TABLE 2
Comparison of model performance

| Model (number of features | | | |
|---|---|---|---|
| selected) | AUROC* | AUPRC | P^{†} |
| Logistic regression (30) | 0.80 (0.76, 0.84) | 0.72 | — |
| Glasgow Coma Scale score (NA) | 0.68 (0.63, 0.73) | 0.61 | <0.001 |
| PRISM IV (NA) | 0.67 (0.62, 0.72) | 0.58 | <0.001 |
| Stepwise Logistic Regression | 0.80 (0.76, 0.84) | 0.74 | 0.834 |
| (16) | | | |
| MARS (14) | 0.90 (0.87, 0.93) | 0.85 | <0.001 |
| Random Forest (30) | 0.91 (0.88, 0.94) | 0.87 | <0.001 |

*(95% confidence intervals);
^{†}vs. logistic regression.
NA, not applicable

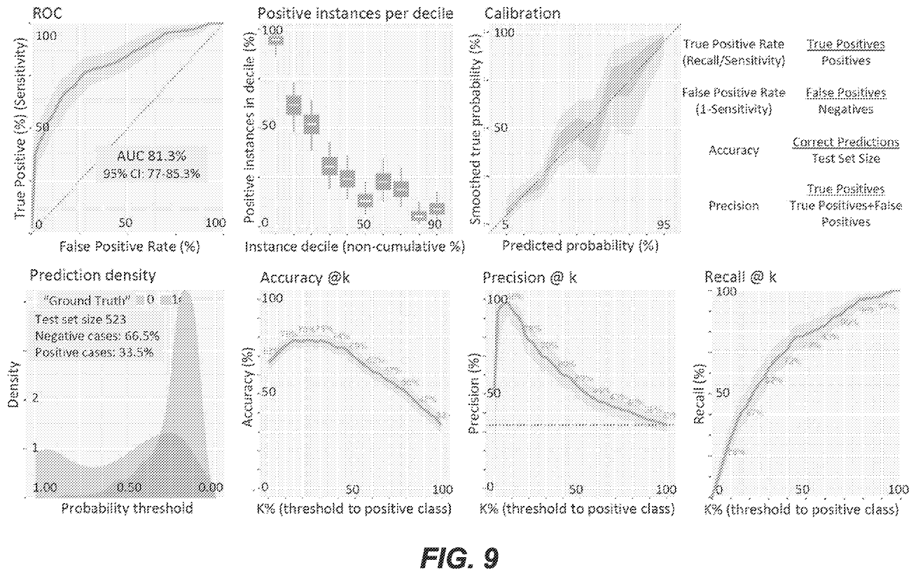

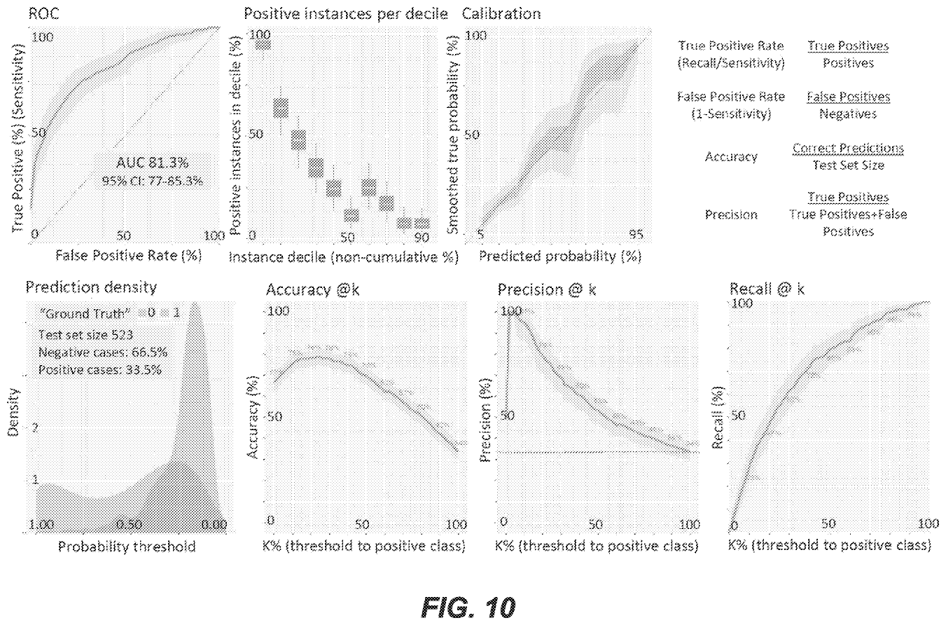

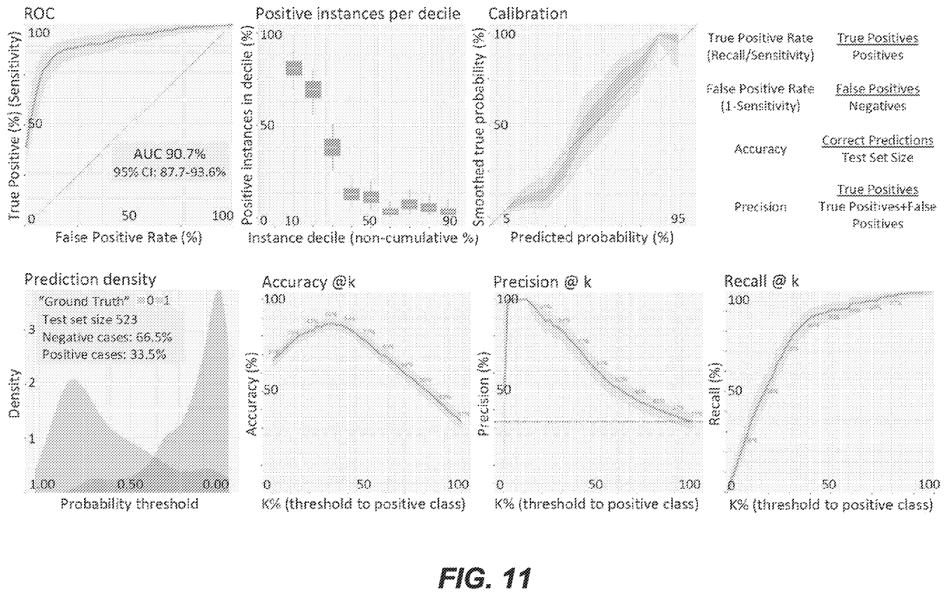

Performance characteristics of the logistic regression model are displayed in FIG. 9. Performance characteristics of the stepwise logistic regression model are displayed in FIG. 10. Performance characteristics of the Random Forest model are displayed in FIG. 11.

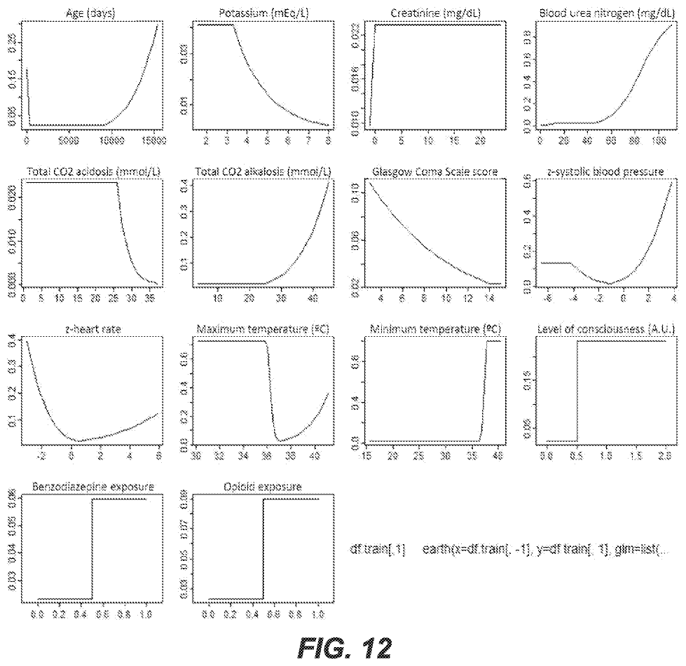

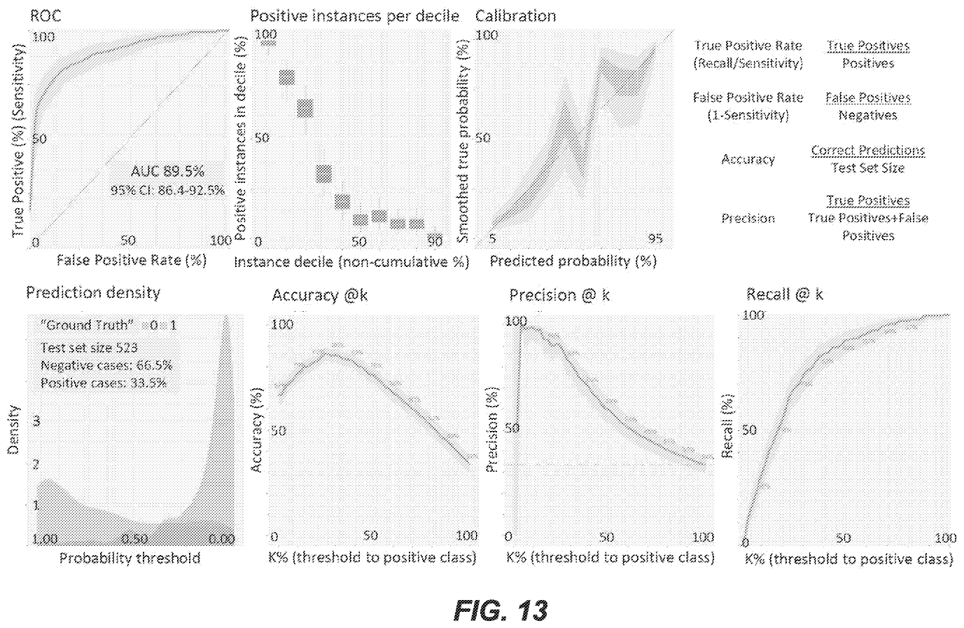

Clinical features used in MARS are shown in FIG. 12. Performance characteristics of the MARS model are shown in FIG. 13.

### Identification of Candidate Brain-Specific Biomarkers

Under an IRB approved protocol, 103 diagnostically diverse PICU patients were prospectively enrolled at the UPMC Children's Hospital of Pittsburgh from November 2012-March 2014. Inclusion criteria included admission to the PICU, <18 years of age, and presence of an indwelling vascular catheter (central venous or arterial) placed as part of routine medical management. Blood was collected on days 1-7 of PICU admission with serum separated and stored at −80° C. for batch analysis.

Biomarkers evaluated included: neuron specific enolase (NSE), an isoenzyme of the glycolytic enzyme enolase that represents functional damage to neurons; myelin basic protein (MBP), part of the myelin sheath of axons; S100B a calcium-binding protein localized in astroglial and Schwann cells utilized for clinical decision making after head trauma in parts of Europe and Asia, and included in the Scandinavian guidelines for initial management of head injuries in adults; ubiquitin carboxyl-terminal hydrolase L1 (UCH-L1), a deubiquitinating enzyme enriched in neurons that is elevated early in serum in adult patients with intracranial injuries and stroke, and in adult patients with septic encephalopathy; glial fibrillary acidic protein (GFAP), an intermediate filament Ill protein found in astrocytes, non-myelinating Schwann cells, and enteric glial cells that is increased after TBI, correlating with abnormal CT findings and in adult patients with septic encephalopathy; and 150 kDa breakdown product of α-II-spectrin (SBDP150), a cytoskeletal component enriched in presynaptic terminals and axons that has been reported to be increased in serum from adult patients with TBI and neonates with congenital heart disease.

A total of 500 serum samples were obtained from the 103 patients enrolled. Primary admission diagnoses included neurological (30.1%), respiratory (19.4%), sepsis/septic shock (12.6%), gastrointestinal/solid organ transplant (11.7%). Twelve patients (11.7%) had unfavorable neurological outcome at hospital discharge. Six patients were deceased (2 declared brain dead, 1 support withdrawn due to suspected poor neurological outcome, and 3 support withdrawn or limitation of care due to medical futility). Both patients declared brain dead were not admitted with a primary neurological diagnosis. NSE, MBP, S100B, UCH-L1, GFAP, and SBDP150 assays were performed using ELISA methods.

In this population, 18/103 (17.5%) developed a new neurological morbidity that included seizure (7/18), structural changes on brain MRI or head CT (11/18; 4 stroke, 5 edema, 3 other), and/or delirium (1/18). Multivariable logistic regression models using peak biomarker concentration of NSE, MBP, S100B, UCH-L1, GFAP, and SBPs for prediction of neurological morbidity are shown in Table 3.

TABLE 3
Biomarkers for validating neurological morbidity

| | | Youden | | | Likelihood |
|---|---|---|---|---|---|
| Biomarker | AUROC* | cutoff | PPV | NPV | ratio |
| NSE | 0.785 (0.855, 0.875) | 7.625^{†} | 0.29 | 0.96 | 2.13 |
| MBP | 0.732 (0.581, 0.883) | 0.455^{†} | 0.37 | 0.93 | 3.08 |
| S100B | 0.63 (0.467, 0.792) | 0.082^{†} | — | — | — |
| UCH-L1 | 0.717 (0.561, 0.872) | 446^{‡} | 0.50 | 0.92 | 5.31 |
| GFAP | 0.749 (0.818, 0.882) | 165.5^{‡} | 0.44 | 0.95 | 4.25 |
| SBDP150 | 0.567 (0.407, 0.728) | 505^{‡} | — | — | — |

*(95% confidence intervals);
^{†}ng/mL;
^{‡}pg/mL

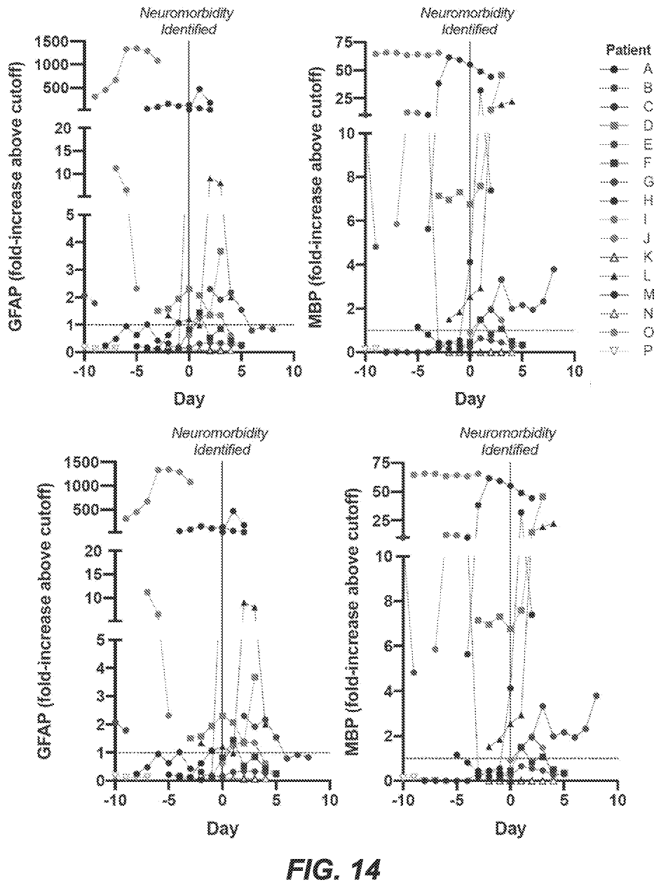

The neuronal biomarkers NSE and UCH-L1, axonal/white matter biomarker MBP, and astrocyte biomarker GFAP were associated with development of hospital-acquired neurological morbidity with AUROC>0.7, favorable negative predictive value (NPV) and likelihood ratios, but positive predictive values (PPV) that are less than optimal. The strengths of the computer algorithm and biomarkers, favorable PPV and NPV, respectively, are complementary for detection of neuromorbidity. Plotting fold-increase above Youden cutoff for UCH-L1, NSE, GFAP, and MBP and the time that neuromorbidity was diagnosed clinically is shown in FIG. 14. These data suggest that strategic use of a biomarker or panel of biomarkers has the capacity to identify neuromorbidity in patients before it is detected clinically. The majority of patients have values above the cutoff in at least one of the biomarkers.

### Method for Detecting and Responding to Neuromorbidity

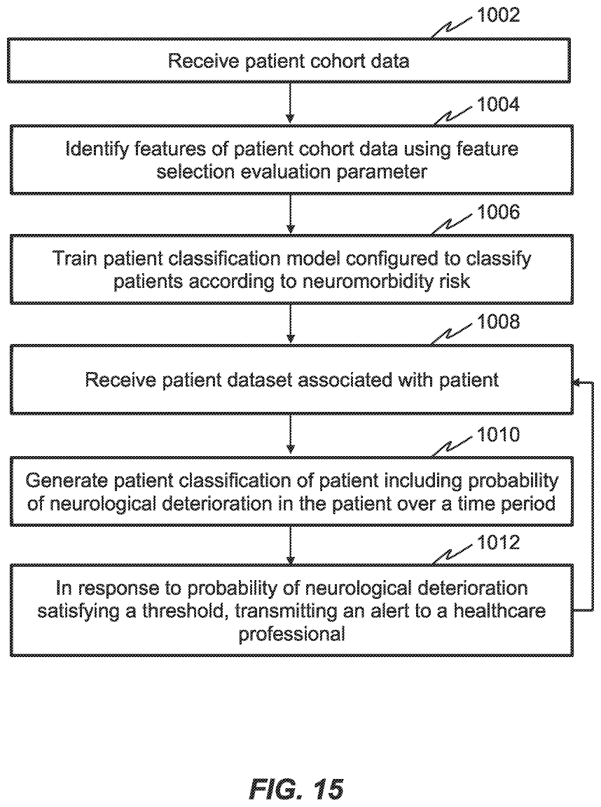

With reference to FIG. 15, provided is a process diagram depicting a method of detecting and responding to patient neuromorbidity according to non-limiting embodiments or aspects. The method may be carried out by one or more computing devices, such as a dedicated server for evaluation of patient data. In step 1002, the server may receive patient cohort data from an electronic health record system. In step 1004, the server may identify features of the patient cohort data using a feature selection evaluating parameter. The feature selection evaluating parameter may include, but is not limited to, one or more of: a first value, a last value, a minimum value, a maximum value, a difference between the minimum value and the maximum value, a difference between the first value and the last value, a change in value over a period of time, a slope of change over a period of time, or a combination thereof. Patient data may include, but is not limited to: level of consciousness or neuro-activity; drugs administered to the patient, including one or more of an opioid, a barbiturate, benzodiazepine, or cisatracurium; bodily fluid parameter(s) or nutrition data, including one or more of glucose, BUN (blood urea nitrogen), z.creatinine (z-scored creatinine); K⁺, min total CO₂, max total CO₂, hypo Na⁺, or hyper Na⁺; hemodynamic status and/or support, including one or more of z.SBP (z-scored systolic blood pressure), z.heart rate, epinephrne, norepinephrine, milrinone, dopamine, dobutamine, or vasopressin; and/or inflammation and/or invasive support, including one or more of min temperature, max temperature, mechanical ventilation, or extracorporeal membrane oxygenation.

In step 1006, the server may train a patient classification model configured to classify patients according to neuromorbidity risk. The patient classification model may include a logistic regression model. The patient classification model may be a machine-learning model, including at least one of the following techniques: Multivariate Adaptive Regression Splines (MARS), random forest, support vector machines, naïve Bayes, and/or the like. The server may convert the features from time series data to vector space representations, in step 1006, prior to training, using the features, the patient classification model. In step 1008, the server may receive a patient dataset associated with a patient. The patient dataset may include, but is not limited to, vital signs of the patient; drugs administered to the patient; pupillary response or reactivity of the patient; at least one bodily fluid parameter of the patient; a Glasgow coma scale score of the patient; a hemodynamic status and/or support; inflammation and/or invasive support; or any combination thereof.

In step 1010, the server may generate, by inputting the patient dataset into the patient classification model, a patient classification of the patient including a probability of the patient developing a neuromorbidity over a time period. In step 1012, in response to the probability of the patient developing a neuromorbidity satisfying a predetermined threshold, the server may transmit an alert (e.g., a text communication, an audio communication, a graphic communication, etc.) to a computing device of a healthcare professional, which may include a physician, a nurse, an advanced practice provider of deteriorating brain health, or any combination thereof.

The server may repeat, at a time interval (e.g., a time period on the order of minutes, hours, and/or the like), the following: receiving a new patient dataset from the record of the patient; generating, by inputting the new patient dataset into the patient classification model, a new patient classification of the patient including a new probability of the patient developing a neuromorbidity over a subsequent time period; and, in response to the new probability of the patient developing a neuromorbidity satisfying the predetermined threshold, transmitting, with at least one processor, the alert to one or more computing devices associated with one or more healthcare professionals, thereby looping through steps 1008, 1010, and 1012. Such a system provides a continuous, updating, real-time monitoring solution to detect early signs of neuromorbidity in patients.

The patient dataset may include a value indicative of levels of at least one brain-specific biomarker. The patient classification, generated in step 1010, may be based at least partly on the value indicative of levels of the at least one brain-specific biomarker. The at least one brain-specific biomarker may include at least one of the following: ubiquitin carboxyl-terminal hydrolase L1 (UCH-L1), glial fibrillary acidic protein (GFAP), myelin basic protein (MBP), neuron specific enolase (NSE), S100b, neurofilament light chain (NFL), Tau, phosphorylated Tau (pTau), cleaved Tau (cTau), 150 kDa breakdown product of α-II-spectrin (SBDP150), or any combination thereof.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A computer-implemented method comprising:
receiving, with at least one processor, patient cohort data from an electronic health record system;
identifying, with at least one processor, features of the patient cohort data using a feature selection evaluating parameter comprising at least one of the following: a first value, a last value, a minimum value, a maximum value, a difference between the minimum value and the maximum value, a difference between the first value and the last value, a change in value over a period of time, a slope of change over a period of time, or a combination thereof;
training, with at least one processor using the features, a patient classification model configured to classify patients according to neuromorbidity risk;
receiving, with at least one processor, a patient dataset associated with a patient;
generating, with at least one processor by inputting the patient dataset into the patient classification model, a patient classification of the patient comprising a probability of the patient developing a neuromorbidity over a time period; and
in response to the probability of the patient developing a neuromorbidity satisfying a predetermined threshold, transmitting, with at least one processor, an alert to a computing device associated with at least one of the following: a physician, a nurse, an advanced practice provider of deteriorating brain health, a health care provider, or any combination thereof.

2. The computer-implemented method of claim 1, wherein the patient dataset comprises at least one of the following: vital signs of the patient; drugs administered to the patient; pupillary response or reactivity of the patient; at least one bodily fluid parameter of the patient; a Glasgow coma scale score of the patient; a hemodynamic status and/or support; inflammation and/or invasive support; or any combination thereof.

3. The computer-implemented method of claim 2, wherein the patient classification model comprises a linear regression model or a logistic regression model.

4. The computer-implemented method of claim 2, wherein the patient classification model comprises a machine-learning model executing at least one of the following techniques: Multivariate Adaptive Regression Splines (MARS); random forest; support vector machines; naïve Bayes; or any combination thereof.

5. The computer-implemented method of claim 1, further comprising converting, with at least one processor, the features from time series data to vector space representations prior to training the patient classification model.

6. The computer-implemented method of claim 1, further comprising, repeating, at a time interval, the following:
receiving, with at least one processor, a new patient dataset from the record of the patient;
generating, with at least one processor by inputting the new patient dataset into the patient classification model, a new patient classification of the patient comprising a new probability of the patient developing a neuromorbidity over a subsequent time period; and
in response to the new probability of the patient developing a neuromorbidity satisfying the predetermined threshold, transmitting, with at least one processor, the alert to the computing device associated with at least one of the following: the physician, the nurse, the advanced practice provider of deteriorating brain health, the health care provider, or any combination thereof.

7. The computer-implemented method of claim 1, wherein the patient dataset comprises a value indicative of levels of at least one brain-specific biomarker, and wherein the patient classification is based at least partly on the value indicative of levels of the at least one brain-specific biomarker.

8. The computer-implemented method of claim 7, wherein the at least one brain-specific biomarker comprises at least one of the following: ubiquitin carboxyl-terminal hydrolase L1 (UCH-L1), glial fibrillary acidic protein (GFAP), myelin basic protein (MBP), neuron specific enolase (NSE), S100b, neurofilament light chain (NFL), Tau, phosphorylated Tau (pTau), cleaved Tau (cTau), 150 kDa breakdown product of α-II-spectrin (SBDP150), or any combination thereof.

9. A system comprising at least one server computer including at least one processor, the at least one server computer programmed and/or configured to:
receive patient cohort data from an electronic health record system;
identify features of the patient cohort data using a feature selection evaluating parameter comprising at least one of the following: a first value, a last value, a minimum value, a maximum value, a difference between the minimum value and the maximum value, a difference between the first value and the last value, a change in value over a period of time, a slope of change over a period of time, or a combination thereof;
train, using the features, a patient classification model configured to classify patients according to neuromorbidity risk;
receive a patient dataset associated with a patient;
generate, by inputting the patient dataset into the patient classification model, a patient classification of the patient comprising a probability of the patient developing a neuromorbidity over a time period; and
in response to the probability of the patient developing a neuromorbidity satisfying a predetermined threshold, transmit an alert to a computing device associated with at least one of the following: a physician, a nurse, an advanced practice provider of deteriorating brain health, a health care provider, or any combination thereof.

10. The system of claim 9, wherein the patient dataset comprises at least one of the following: vital signs of the patient; drugs administered to the patient; pupillary response or reactivity of the patient; at least one bodily fluid parameter of the patient; a Glasgow coma scale score of the patient; a hemodynamic status and/or support; inflammation and/or invasive support; or any combination thereof.

11. The system of claim 9, wherein the at least one server computer is further programmed and/or configured to convert the features from time series data to vector space representations prior to training the patient classification model.

12. The system of claim 9, wherein the at least one server computer is further programmed and/or configured to repeat, at a time interval, the following:
receiving a new patient dataset from the record of the patient;
generating, by inputting the new patient dataset into the patient classification model, a new patient classification of the patient comprising a new probability of the patient developing a neuromorbidity over a subsequent time period; and
in response to the new probability of the patient developing a neuromorbidity satisfying the predetermined threshold, transmitting the alert to the computing device associated with at least one of the following: the physician, the nurse, the advanced practice provider of deteriorating brain health, the health care provider, or any combination thereof.

13. The system of claim 9, wherein the patient dataset comprises a value indicative of levels of at least one brain-specific biomarker, and wherein the patient classification is based at least partly on the value indicative of levels of the at least one brain-specific biomarker.

14. The system of claim 13, wherein the at least one brain-specific biomarker comprises at least one of the following: ubiquitin carboxyl-terminal hydrolase L1 (UCH-L1), glial fibrillary acidic protein (GFAP), myelin basic protein (MBP), neuron specific enolase (NSE), S100b, neurofilament light chain (NFL), Tau, phosphorylated Tau (pTau), cleaved Tau (cTau), 150 kDa breakdown product of α-II-spectrin (SBDP150), or any combination thereof.

15. A computer program product comprising at least one non-transitory computer-readable medium including program instructions that, when executed by at least one processor, cause the at least one processor to:
receive patient cohort data from an electronic health record system;
identify features of the patient cohort data using a feature selection evaluating parameter comprising at least one of the following: a first value, a last value, a minimum value, a maximum value, a difference between the minimum value and the maximum value, a difference between the first value and the last value, a change in value over a period of time, a slope of change over a period of time, or a combination thereof;
train, using the features, a patient classification model configured to classify patients according to neuromorbidity risk;
receive a patient dataset associated with a patient;
generate, by inputting the patient dataset into the patient classification model, a patient classification of the patient comprising a probability of the patient developing a neuromorbidity over a time period; and
in response to the probability of the patient developing a neuromorbidity satisfying a predetermined threshold, transmit an alert to a computing device associated with at least one of the following: a physician, a nurse, an advanced practice provider of deteriorating brain health, a health care provider, or any combination thereof.

16. The computer program product of claim 15, wherein the patient dataset comprises at least one of the following: vital signs of the patient; drugs administered to the patient; pupillary response or reactivity of the patient; at least one bodily fluid parameter of the patient; a Glasgow coma scale score of the patient; a hemodynamic status and/or support; inflammation and/or invasive support; or any combination thereof.

17. The computer program product of claim 15, wherein the program instructions further cause the at least one processor to convert the features from time series data to vector space representations prior to training the patient classification model.

18. The computer program product of claim 15, wherein the program instructions further cause the at least one processor to repeat, at a time interval, the following:
receiving a new patient dataset from the record of the patient;
generating, by inputting the new patient dataset into the patient classification model, a new patient classification of the patient comprising a new probability of the patient developing a neuromorbidity over a subsequent time period; and
in response to the new probability of the patient developing a neuromorbidity satisfying the predetermined threshold, transmitting the alert to the computing device associated with at least one of the following: the physician, the nurse, the advanced practice provider of deteriorating brain health, the health care provider, or any combination thereof.

19. The computer program product of claim 18, wherein the patient dataset comprises a value indicative of levels of at least one brain-specific biomarker, and wherein the patient classification is based at least partly on the value indicative of levels of the at least one brain-specific biomarker.

20. The computer program product of claim 19, wherein the at least one brain-specific biomarker comprises at least one of the following: ubiquitin carboxyl-terminal hydrolase L1 (UCH-L1), glial fibrillary acidic protein (GFAP), myelin basic protein (MBP), neuron specific enolase (NSE), S100b, neurofilament light chain (NFL), Tau, phosphorylated Tau (pTau), cleaved Tau (cTau), 150 kDa breakdown product of α-II-spectrin (SBDP150), or any combination thereof.

21. A method of treating a patient having increased risk of development of a neuromorbidity, comprising:
receiving, from a computing device comprising the computer program product of claim 15, the patient classification of the patient or the alert; and
increasing monitoring of the patient for development of the neuromorbidity and/or treating the patient for the neuromorbidity when the patient is classified as having increased risk of developing a neuromorbidity or an alert is transmitted indicating the patient as having increased risk of developing a neuromorbidity.
